(19) United States
(12) Patent Application Publication
(10) Pub. No.: US 2010/0168601 A1
(43) Pub. Date: Jul. 1, 2010

(54) COMBINED CANNULA AND AIRFLOW TEMPERATURE SENSOR AND THE METHOD OF USING THE SAME

(75) Inventors: Kyle ADRIANCE, Bakersfield, CA (US); James CHUA, Bakersfield, CA (US); James N. CURTI, Bakersfield, CA (US); Eric C. LAND, Bakersfield, CA (US)

Correspondence Address: DAVIS & BUJOLD, P.L.L.C., 112 PLEASANT STREET, CONCORD, NH, 03301, US

(73) Assignee: SALTER LABS, Arvin, CA (US)

(21) Appl. No.: 12/647,070

(22) Filed: Dec. 24, 2009

Related U.S. Application Data
(63) Continuation-in-part of application No. 12/134,787, filed on Jun. 6, 2008.
Application No. 12/134,787 is a continuation-in-part of application No. 12/348,599, filed on Jan. 5, 2009.
Application No. 12/348,599 is a continuation-in-part of application No. 61/174,704, filed on May 1, 2009.

(51) Int. Cl. A61B 5/08

(52) U.S. Cl. 600/537; 600/529

(57) ABSTRACT

A combined cannula and temperature sensing device for detecting breathing of a patient. The cannula comprising a cannula body defining at least one internal flow chamber therein, at least one tube being connected to the cannula body and communicating with the internal flow chamber, a pair of nasal prongs communicating with the internal flow chamber, and the cannula body having retaining mechanism for releasably attaching the temperature sensing device to the cannula. The temperature sensing device comprising a sensor body, at least one temperature sensor being supported by the sensor body, and the sensor body releasably engaging with the retaining mechanism of the cannula for positioning the at least one temperature sensor adjacent at least one of a nasal cavity and a mouth of the patient for detecting breathing of the patient.

### FIELD OF THE INVENTION

The present invention relates to a sleep monitoring and diagnosing system including an air temperature sensing device which is suitable for monitoring nasal and/or oral breath by obtaining respiratory air wave and airflow information during a sleep apnea diagnostic session and processing the acquired air wave and airflow breathing information and inputting the same to conventional polysomnography equipment.

### BACKGROUND OF THE INVENTION

Sleep apnea (SA) is a common disorder observed in the practice of sleep medicine and is responsible for more mortality and morbidity than any other sleep disorder. Sleep apnea is characterized by recurrent failures to breathe adequately during sleep (termed apneas or hypopneas) as a result of obstructions in the upper airway.

Apnea is typically defined as a complete cessation of airflow. Hypopnea is typically defined as a reduction in airflow disproportionate to the amount of respiratory effort expended and/or insufficient to meet the individual's metabolic needs. During an apnea or hypopnea-commonly referred to as a respiratory event-oxygen levels in the brain decrease while the carbon dioxide (CO₂) levels rise, thereby causing the person sleeping to awaken. The heart beats rapidly and blood pressure rises to levels (up to 300 mm Hg). The brief arousals to breathe are followed by a return to sleep, but in severe cases the apneas may recur over 60 times per hour.

Sleep apnea is a serious, yet treatable health problem for individuals. Published reports indicate that untreated sleep apnea patients are three to five times more likely to be involved in industrial and motor vehicle accidents that involve impaired vigilance and memory. Studies show that more than 15% of men and 5% of women over the age of 30 and up to 30% of men and women over the age of 65 suffer from sleep apnea. Sleep apnea during pregnancy is associated with hypertension and a risk of growth retardation of the fetus. Current estimates reveal that over 90% of individuals, with moderate to severe sleep apnea, remain undiagnosed.

The current standard for the diagnosis of sleep apnea is called polysomnography (PSG), which is administered and analyzed by a trained technician and reviewed by a Board Certified Sleep Specialist. The limited availability of sleep centers coupled with the high capital expense, in order to add additional capacity for diagnosis of sleep disorders, has resulted in a growing number of patients awaiting analysis by PSG.

A conventional full overnight PSG includes recording of the following signals: electroencephalogram (EEG), sub-mental electromyogram (EMG), electroculogram (EOG), respiratory airflow (oronasal flow monitors), respiratory effort (plethysmography), oxygen saturation (oximetry), electrocardiography (ECG), snoring sounds and body position. These signals are considered the “gold standard” for the diagnosis of sleep disorders in that they offer a relatively complete collection of parameters from which respiratory events may be identified and sleep apnea may be reliably diagnosed. The RR interval, commonly referred to as beats per minute, is derived from the ECG. The body position is normally classified as: right side, left side, supine, prone, or up (e.g., sitting erect). Typically, a microphone is taped over the pharynx and the body position sensor is attached over the sternum of the patient's chest. Each signal provides some information which assists with the visual observation and recognition of the respiratory events.

A collapse of the upper airway is identified when the amplitude of the respiratory airflow and the effort signals decrease by at least 50%, snoring sounds either crescendo or cease, and oxygen desaturation occurs. A respiratory event is confirmed (i.e., desaturation not a result of artifact) by the recognition of an arousal (i.e., the person awakens to breathe), typically identified by an increase in the frequency of the EEG, an increase in the heart rate or changing in snoring pattern. The remaining signals assist in determining specific types of respiratory events. For example, the EEG and EOG signals are used to determine if a respiratory event occurred in either non-rapid eye movement (NREM) or rapid eye movement (REM) sleep. The position sensor is used to determine if an airway collapse occurs only, or mostly, in just one body position (e.g., typically the supine position).

A reduction or absence of airflow at the airway opening defines sleep-disordered breathing. In an adult, the absent of airflow for a duration of 10 seconds is apnea, and airflow reduced below a certain amount is a hypopnea. Ideally one would measure actual flow with a pneumotachygraph of some sort, but in clinical practice this is impractical, and devices that are comfortable and easy to use are generally substituted. The most widely used are thermistors which are placed in front of the nose and the mouth to detect temperature changes of a thermally sensitive resistor, e.g., heating (due to expired gas) and cooling (due to inspired air). They provide recordings of changes in airflow, but as typically employed are not quantitative instruments. Currently available thermistors are sensitive, but frequently lag or have a delay in response time relative to pressure sensors and pressure transducers. Also, if they touch the skin, they normally cease being flow sensors. In some laboratories, measurement of end tidal CO₂ is used to detect expiration to produce both qualitative and quantitative measures of a patient's breath.

An alternative method is to measure changes in pressure in the nasal airway that occur during breathing. This approach provides an excellent reflection of true nasal flow. A simple nasal cannula attached to a pressure transducer can be used to generate a signal that resembles one obtained with a pheumatachygraph. It allows detection of the characteristic plateau of pressure due to inspiratory flow limitation that occurs in subtle obstructive hypopneas.

An obstructive apnea or hypopnea is defined as an absence or reduction in airflow, in spite of continued effort to breathe, due to obstruction in the upper airway. Typical polysomnography includes some recording of respiratory effort. The most accurate measure of the effort is a change in pleural pressure as reflected by an esophageal pressure monitor. Progressively more negative pleural pressure swings, leading to an arousal, have been used to define a “Respiratory Effort Related Arousal” (RERA), the event associated with the so-called “Upper Airway Resistance Syndrome”. However the technology of measuring esophageal pressure is uncomfortable and expensive, and rarely used clinically. Most estimates of respiratory effort, during polysomnography, depend on measures of rib cage and/or abdominal motion. The methods include inductance or impedance plethysmography, or simple strain gages. Properly used and calibrated, any of these devices can provide quantitative estimates of lung volume and abdominal-rib cage paradox. However, calibrating during an overnight recording is very difficult and, as a practical matter, is almost never done. The signals provided by respiratory system motion monitors are typically just qualitative estimates of respiratory effort.

Pressure sensing devices are currently available and used during a sleep diagnostic session to detect changes in respiratory air pressure and/or airflow to confirm whether or not a patient is breathing and to gather other breathing information from the patient. Accurate modeling of the patient's breathing cycle is limited by the use of only pressure sensors as the placement of sensors and system failures can cause false readings or pressure offsets that must be adjusted or compensated in order to properly model the breathing cycle.

Combining pressure sensor measurements with temperature sensor measurements can improve breath monitoring and modeling thereby leading to a more accurate diagnosis and more quickly determine a patient's breathing failure by utilizing temperature monitors directly positioned at the nasal and the oral breathing passages of the patient. Additionally, in using a temperature sensor for breath monitoring, it is generally necessary to test the electrical leads and circuit components of the temperature sensing device to insure that all of the electrical leads and components are, in fact, operational and not faulty.

In addition, conventional test circuitry typically is completely separate from the temperature sensing device and this leads to further difficulties such as the test circuitry being either misplaced, lost, having insufficient electrical power, etc., thereby rendering it difficult to test the pressure sensing device prior or during use.

### SUMMARY OF THE INVENTION

It is an object of the invention to provide a system including an apparatus and method for monitoring patient breathing by use of a temperature sensor adapted for use alone on a patient or possibly for use in combination with either a nasal cannula or a combined nasal/oral cannula.

Another object of the invention is to generate accurate wave forms for tracing respiratory events of patient breathing so that reliable and accurate signals can be sent to a headbox of virtually any PSG records currently available.

A further object of the invention is to provide a monitoring apparatus for patient breathing which self generates a signal and thereby avoids the need for an external power source.

A still further object of the invention is to provide an apparatus and method for monitoring patient breathing which is disposable while, at the same time, still being convenient, cost effective and more comfortable and less stress for the patient to wear.

Yet another object of the invention is to provide an apparatus and method for monitoring patient breathing which is available in various sizes (e.g., adult, children and pediatric) and is readily connectable with conventional adaptor cables.

It is a further object of the invention to provide a method of securing a sensor body of the temperature sensor in a temperature sensing position, on an upper lip of the patient, without the sensor body having a tendency to “roll” or otherwise move, turn or wobble significantly, during a sleep diagnostic session, so that each one of the temperature probes can be directly located in the airflow exhausting from and/or being supplied to one of the nostrils or the mouth of the patient while constantly maintaining each of the temperature probes of the temperature sensor out of contact with the skin of the patient, during the entire sleep diagnostic session, to avoid obtaining any false reading or measurements.

Another object of the invention is to provide an electronic circuit for the temperature sensors that has connections to an external microprocessor or controller to measure and accurately model a patient's breathing patterns based on the temperature and pressure data (e.g., sense oral/nasal thermal airflow changes) so as to provide a diagnosis for sleep apnea or, alternatively, to provided a basis for determining proper gas and oxygen delivery to a patient.

Another object of the present invention is to design the temperature sensing device so that the sensor body, of the temperature sensing device, facilitates secure mounting of the temperature sensing device to the patient while still permitting any desired adjustment of each of the temperature sensors so that they properly align with either the nasal and oral expiration and inspiration, i.e., air flow, of the patient.

The present invention relates to a sensor body incorporating at least one airflow sensing device for receiving respiratory breathing information from a patient to be monitored. The sensor body generally having a T-shape configuration.

Preferably, the sensor body is shaped and curved in two planes which will allow the sensor body to rest securely and comfortably, on the upper lip of a patient, without the need to use adhesive tape to stabilize the sensor body to prevent the sensor body from rolling, twisting, wobbling or the like. It is critically important that the tips of each one of the temperature sensors (that is both of the nasal sensors and the oral sensor) remain in their originally aligned position, so that any changes in the patient's breathing can be easily detected, in the airflow waveform, during a sleep diagnostic session. If and when the airflow changes, as it will normally occur during obstructive sleep apnea, the clinical operator needs to be confident that such changes reflect the quality of breath and are not due to some mal-positioning of one of the airflow temperature sensors, e.g., one of the temperature contacting the skin of the patient.

The present invention relates to a sensor body which supports at least one temperature sensor, such as a thermocouple or a thermistor, and adequately spaces the temperature sensor(s) from the skin of a patient so that at least one temperature sensor(s) is adequately supported by the upper lip, adjacent to the nostril and/or mouth of the patient, and adequately positioned to detect breathing of the patient, without the temperature sensor(s) being prone to contact the skin of the patient during the sleep diagnostic session.

The present invention also relates to a T-shaped configuration in which each branch of the T-shaped configuration has a respective temperature sensor for detecting the flow from an oral or nasal cavity of the patient with each of the temperature sensors being sufficiently from the skin of the patient, by the sensor body, so that each of the temperature sensors avoids contact the skin of the patient during the sleep diagnostic session.

A still further object of the present invention is to provide a sensor body which facilitates coupling of the temperature sensing device to a cannula so that the temperature sensing device maybe directly supported by, but spaced from the face of the patient, the nasal cannula with each of the temperature sensors still being positioned adjacent one of the nasal or oral cavities of the patient so that each of the temperature sensors can adequately sense breathing of the patient.

Still further object of the present invention is to provide a disposable combined temperature sensing device/cannula assembly in which the temperature sensing device is supported by the cannula, during the sleep diagnostic session, so as to prevent the temperature sensor from contacting the face of the skin, and following completion of the sleep diagnostic session, the entire temperature sensing device/cannula assembly is properly disposed of.

Yet another object of the present invention is to provide a relatively “permanent” temperature sensing device which can be removably supported by a cannula, during the sleep diagnostic session, so as to prevent the temperature sensor from contacting the face of the skin, but following completion of the sleep diagnostic session, the temperature sensing device can be removed from the cannula for reuse while the cannula is properly disposed of.

### BRIEF DESCRIPTION OF THE DRAWINGS

The invention will now be described, by way of example, with reference to the accompanying drawings in which:

FIG. 1 is a front elevational view of a temperature sensor device having a pair of spaced apart nasal airflow temperature sensors and an oral airflow temperature sensor, according to the present invention;

FIG. 2 is a top view of FIG. 1;

FIG. 3 is a bottom plan view of FIG. 1;

FIG. 4 is a left side elevational view of FIG. 1

FIG. 5 is an illustrative wiring diagram of the temperature sensors of FIG. 1;

FIG. 5A is an alternative diagrammatic wiring diagram for the temperature sensors of FIG. 1;

FIG. 6 is a diagrammatic wiring diagram for the temperature sensors of FIG. 1;

FIG. 7 is a front elevational view of another embodiment of the sensor body only having an oral airflow temperature sensor;

FIG. 8 is a front elevational view of a still further embodiment of the sensor body only having a pair of spaced apart nasal airflow temperature sensors;

FIG. 9 is a front elevational view of a second embodiment of the temperature sensor according to the present invention;

FIG. 10 is a bottom, right, front perspective view of an undivided cannula for use in supporting the temperature sensor according to the present invention;

FIG. 11 is a front elevational view of the cannula of FIG. 10 for use in supporting the temperature sensor according to the present invention;

FIG. 12 is a right side elevational view of the temperature sensor, according to the present invention, used in combination with the cannula of FIG. 10;

FIG. 13 is a front elevational view of a variation of the cannula of FIG. 10 for use in supporting the temperature sensor device according to the present invention;

FIG. 14 is a diagrammatic view of a divided cannula for use in supporting the temperature sensor device according to the present invention;

FIG. 15 is a diagrammatic view of an undivided cannula with a single oral sensing prong for use in supporting the temperature sensor device according to the present invention;

FIG. 16 is a diagrammatic view of a divided cannula with a single oral sensing prong for use in supporting the temperature sensor device according to the present invention;

FIG. 17 is a diagrammatic view of an undivided cannula with dual oral sensing prongs for use in supporting the temperature sensor device according to the present invention;

FIG. 18 is a diagrammatic view of a divided cannula having dual oral sensing prongs for use in supporting the temperature sensor device according to the present invention;

FIG. 19 is a diagrammatic view of a divided cannula having dual oral sensing prongs with three separate gas supply/sensing flow paths for use in combination with the temperature sensor device according to the present invention;

FIG. 20 is a diagrammatic view of a divided cannula having dual oral sensing prong with four separate gas supply/sensing flow paths for use in combination with the temperature sensor device according to the present invention;

FIG. 21 is a front elevational view of another embodiment of an undivided cannula for use in supporting the temperature sensor, according to the present invention, having an oral prong defining dual passageways therein;

FIG. 22 is a diagrammatic left side elevational view of FIG. 21;

FIG. 23 is a diagrammatic top plan view of FIG. 21;

FIG. 24 is a diagrammatic bottom plan view of FIG. 21;

FIG. 25 is a front elevational view of a further embodiment of an undivided cannula for use in supporting the temperature sensor, according to the present invention, having an oral prong with only a single passageway therein;

FIG. 26 is a front elevational view of another embodiment of an undivided cannula for use in supporting the temperature sensor, according to the present invention, having an oral prong defining dual passageways therein and a single horizontally arranged holster;

FIG. 27 is a diagrammatic left side elevational view of FIG. 26;

FIG. 28 is a diagrammatic top plan view of FIG. 26;

FIG. 29 is a diagrammatic bottom plan view of FIG. 26;

FIG. 30 is a front elevational view of another embodiment of an undivided cannula for use in supporting the temperature sensor, according to the present invention, having an oral prong defining dual passageways therein and a pair of spaced apart holsters;

FIG. 31 is a diagrammatic left side elevational view of FIG. 30;

FIG. 32 is a diagrammatic top plan view of FIG. 30;

FIG. 33 is a diagrammatic bottom plan view of FIG. 30;

FIG. 34 is a front elevational view of a reusable temperature sensor device having a pair of spaced apart nasal airflow temperature sensors and an oral airflow temperature sensor;

FIG. 35 is a top view of FIG. 34; and

FIG. 36 is a diagrammatic front elevational view of a variation of the temperature sensor device for an infant application.

### DETAILED DESCRIPTION OF THE INVENTION

The present invention is directed to an apparatus and a method for monitoring and modeling a patient's breathing according to temperature measurements obtained during either an exhalation and/or an inhalation interval of a patient during a sleep diagnostic session. A temperature sensor-typically a thermocouple although other types of temperature sensors such as a thermistor, etc., could be used as well-is positioned adjacent the nares (nostrils) of the patient's nose (for nasal temperature sensing) and adjacent the patient's mouth (for oral temperature sensing). The obtained output signals, from the temperature sensor(s) which are then processed into the acquired air wave and airflow breathing data for input to conventional polysomnography equipment which produces an output representation of the patient's breathing cycle generally as a qualitative, viewable waveform.

It is preferable to measure temperature changes during the exhalation and the inhalation interval using a plurality of temperature sensors. For example, accurate temperature measurements are made with a temperature sensor positioned adjacent each of the left and the right nasal cavities as well as the mouth of the patient. However, in some applications it is possible to acquire fairly accurate temperature measurements via only a single temperature sensor positioned adjacent the patient's mouth (see FIG. 7) or possibly with only a pair of temperature sensors positioned adjacent each of the nasal cavities of the patient's nose (see FIG. 8).

When compared with typical pressure sensing devices, the temperature sensors can be beneficial. If a patient breathes exclusively through his/her mouth, it is often difficult to obtain an accurate pressure measurement based on inspiration and/or expiration through the mouth. The size of a patient's mouth in general can make it somewhat challenging to align precisely a cannula opening, of an oral prong, at a suitable position in order to sense oral inspiration and oral expiration. For example, a person may breathe out the side of his/her mouth and thus an oral prong, located in the center of the mouth for pressure sensing, may not receive adequate breathing flow in order to properly determine pressure variations. In the case of a mouth breather like this, a temperature sensor, such as a thermocouple (e.g., a junction between two different metals that produces a voltage related to a temperature difference) or a thermistor, typically provides the best response to the temperature differential it experiences between ambient air and whatever portion of the patient's breathing which interacts with the temperature sensor.

In general, and as discussed in further detail below, in order to most effectively determine an accurate wave form including the most accurate amplitude as well as frequency, i.e., breaths per minute, of actual inspiration and expiration of a patient, the preferred embodiment of the system has at least one, more preferably two, and most preferably three temperature sensors, such as thermocouples or thermistors, as the temperature sensors for detecting the oral and/or the nasal temperature changes induced by a patient's inspiration and expiration. As noted above, the temperature sensing device may be releasably affixed to and carried by and used in combination with a desired nasal cannula or a desired nasal/oral cannula or, if so desired, the sensor body may be directly affixed to and supported by the upper lip of a patient. The airflow sensing device obtains desired nasal and/or oral airflow information which assists with precisely monitoring, modeling and diagnosing a patient's respiratory airflow and breathing cycles which assists with facilitating confirmation of distress signals from hypopneas, apnea and other sleep events.

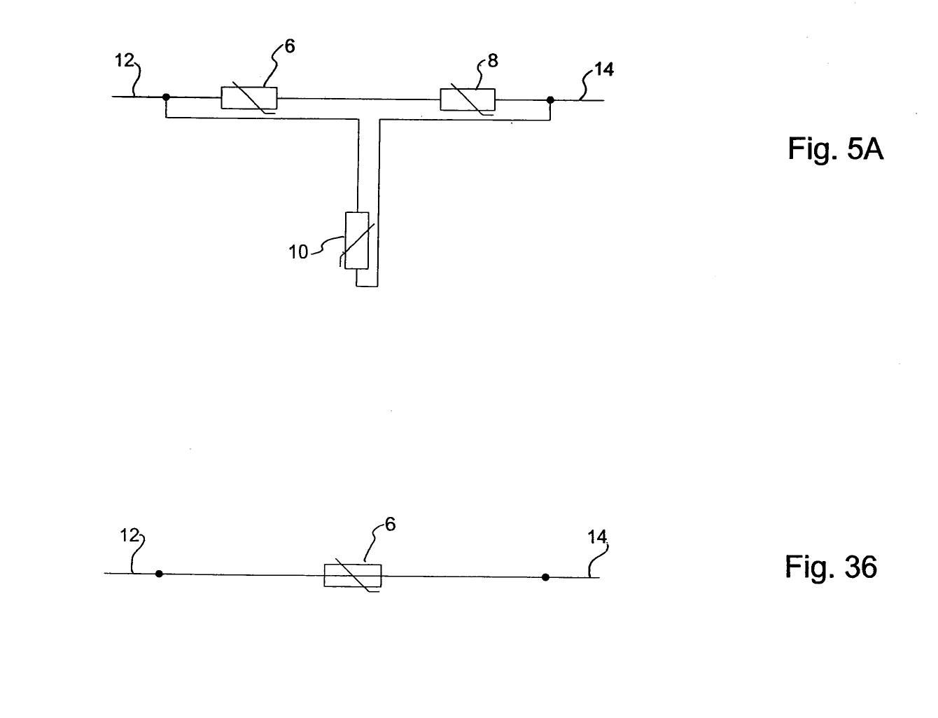

Turning now to the FIGS. 1-5, the improved temperature sensing device 2, according to the present invention, generally comprises a sensor body 4 which supports a first nasal temperature sensor 6, a second nasal temperature sensor 8, and a third oral temperature sensor 10. The three separate temperature sensors 6, 8, 10, e.g., generally three thermocouples or possibly thermistors, are each connected in series with one another, as diagrammatically shown in FIG. 5. Alternatively, the first and the second temperature sensors 6 and 8, e.g., either thermocouples or thermistors, are each connected in series with one another, as diagrammatically shown in FIG. 5A, while the third temperature sensor 10, e.g., either a thermocouple or a thermistor, is connected in parallel with both the first and the second temperature sensors 6 and 8.

The sensor body 4 generally has a T-shaped configuration having a left first branch 16, a right second branch 18 and a central third branch 20. The nasal first temperature sensor (e.g., thermocouple) 6 is embedded or located within the first branch 16, the nasal second temperature sensor (e.g., thermocouple) 8 is embedded or located in the second branch 18 and the oral third temperature sensor (e.g., thermocouple) 10 is embedded or supported by and extends from a lower end of the third branch 20. As shown in FIGS. 1-4, each of the first and the second nasal temperature sensors (e.g., thermocouple) 6, 8 extend a distance of about ⅛ of an inch to about ½ inch generally normal to the sensor body 4 and more preferably about ¼ of an inch or so from the sensor body 4 while the oral third temperature sensor (e.g., thermocouple) 10 extends a distance of about ½ of an inch to about 2.5 inches from the lower end of the central branch 20 and more preferably about 1 inches from the lower end of the central branch 20. Once properly positioned on the face of a patient, the left first and the right second branches 16, 18 each extend in a lateral direction, above the upper lip but below the nasal septum of the nose of the patient, each generally extending toward one of the ears of the patient while the third temperature sensor 10 is generally positionable adjacent and centered with respect to the mouth of the patient. As a result of such arrangement, each of the nasal first and second temperature sensors 6, 8 are readily positionable generally adjacent and centered with respect to a respective nasal cavity, e.g., partially within one of the left and the right nostrils of the patient, while the third temperature sensor is generally centered, with respect to the mouth of the patient, so that it can be easily manipulated into a desired sensing position.

The T-shaped sensor body 4 is typically manufactured from a material which does not cause any itching, irritation and/or rash to the skin of the patient so that the temperature sensing device 2 may be worn by the patient for a prolong period of time, e.g., placed directly on the upper lip of the patient at least overnight during a sleep diagnostic session. This minimizes the possibility of the patient inadvertently tugging or altering the installed position of the temperature sensing device 2 during the sleep diagnostic session. Suitable materials for manufacture of the sensor body 4 include, for example, silicone, polyvinyl chloride (PVC) and thermoplastic elastomer (TPE).

The sensor body 4 generally has a maximum thickness or diameter of between about 0.1 and about 0.25 inches and more preferably has a maximum thickness or diameter of about 0.15 inches such that the sensor body 4 sufficiently spaces each one of the temperature sensors 6, 8 and 10 from the skin of the patient, during use, so as to avoid contact by any of the temperature sensors 6, 8 and 10 with the skin of the patient. Although the sensor body 4 is shown as generally having a cylindrical transverse cross sectional shape, it is to be appreciated that other transverse cross sectional shapes, e.g., oval, square, rectangular, triangular, hexagonal, etc., can be used and, for such embodiments the sensor body 4 may have a maximum thickness or diameter of between about 0.1 and about 0.375 or so whereby the sensor body 4 facilitates adequately spacing of each of the temperature sensors 6, 8, 10 from the face of the patient and/or facilitates secure retention of the temperature sensing device 2, at least during a sleep diagnostic session, either directly to the face of a patient or to a desired cannula.

Each of the first branch 16, the second branch 18 and the third branch 20 of the temperature sensing device 2 are joined with one another by a central hub 26 of the sensor body 4. The first branch 16 extends from the central hub 26 by a distance of between about ¼ and about 2 inches, more preferably by a distance of about ½ inch and about 1¼ inches, and most preferably by a distance of about ¾ inch or so. The second branch 18 extends from the central hub 26 by a distance of between about ¼ and about 2 inches, more preferably by a distance of about ½ inches and about 1¼ inches, and most preferably by a distance of about ¾ inch or so. The third branch 20 extends from the central hub 26 by a distance of between about 1/16 and about ¾ inches, more preferably by a distance of about ⅛ inches and about ½ inches, and most preferably by a distance of about a distance of ¼ of an inch or so.

The nasal first temperature sensor (e.g., thermocouple) 6 extends from the first branch 16, generally midway between the central hub 26 and a remote end 22 of the first branch 16, the nasal second temperature sensor (e.g., thermocouple) 8 extends from the second branch 18, generally midway between the central hub 26 and a remote end 24 of the second branch 18, and the oral third temperature sensor (e.g., thermocouple) 10 extends from the third branch 20 generally from a center of a remote end 28 of the third branch 20 so as to facilitate positioning at a desired location with respect to the oral cavity of the patient.

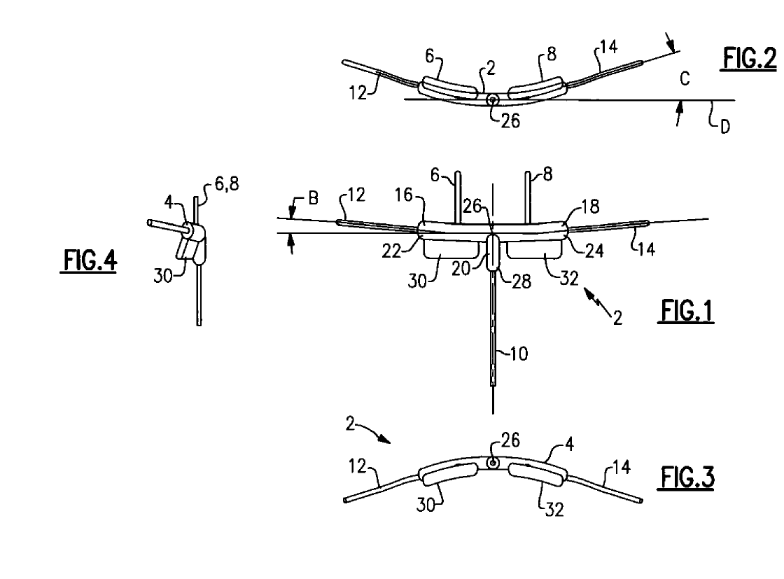

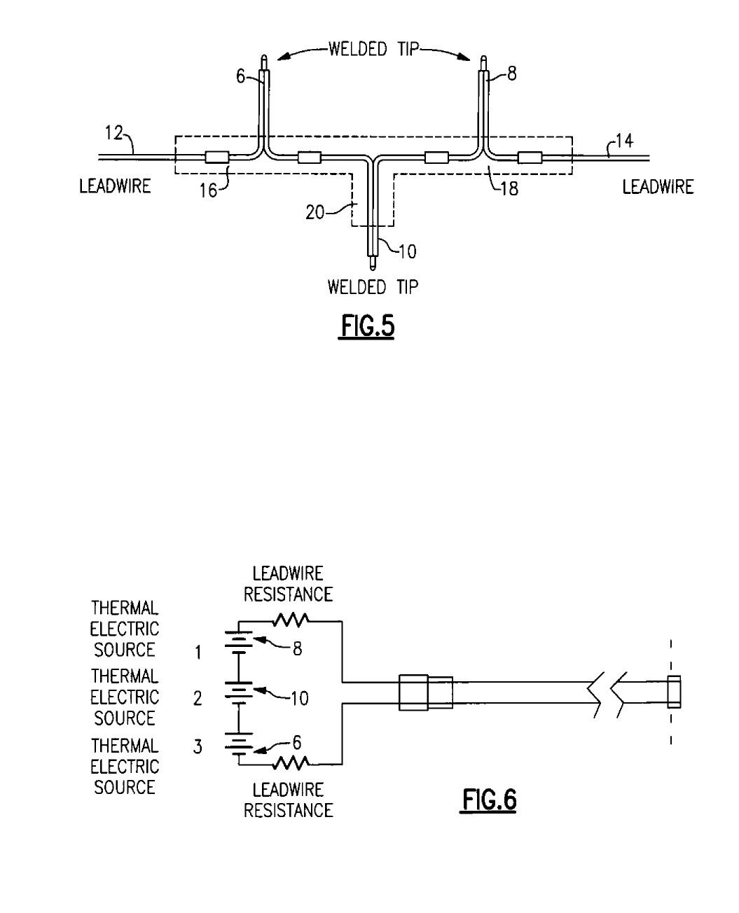

In a preferred form of the sensor body 4, the left and the right branches 16, 18 are molded and curved so as to form a slightly upward acute angle B with respect to a horizontal plane A passing horizontally through the central hub 26 and normal to the third branch 20 of the sensor body 4, as shown in FIGS. 1, 7 and 8, such that the remote ends 22, 24 of the left and the right branches 16, 18 preferably curve and extend slightly upward generally toward the patient's ears, once the temperature sensing device 2 is installed above the upper lip of the patient. The left and the right branches 16, 18 are also molded and curved so as to form a rearward acute angle C, as shown in FIGS. 2 and 3, with respect to a vertical plane D passing through the central hub 26 and generally parallel to the third branch 20 of the sensor body 4 such that the left and right branches 16, 18 also bend or extend rearward toward the patient's ears, once the temperature sensing device 2 is installed above the upper lip of the patient.

The above configuration of the left and right branches 16, 18 together with the lower branch 20 serve to form a stable base or support for the temperature sensing device 2, on the face of a patient, which minimizes any rocking, rolling, pivoting, wobbling and/or other undesired motion of the temperature sensing device 2, during a sleep diagnostic session, which may have a tendency to cause any of the temperature sensors 6, 8, 10 to contact inadvertently the skin of the patient or move out of the air stream. The novel design of the temperature sensing device 2 is directed at maintaining the initially installed orientation of the temperature sensing device 2 substantially constant during the entire diagnostic session.

The upward acute angle B, formed between the left and right branches 16, 18 and the horizontal plane A, is preferably between about 2° and 45° and more preferably between about 4° and 30° and most preferably about 5°. The rearward acute angle C, formed between the left and right branches 16, 18 and the vertical plane D that passes through the central hub 26 of the temperature sensing device 2, is preferably between about 5° and 40° and more preferably between about 10° and 25° and most preferably about 17.5°. Such range of the acute angles B and C both facilitate angular separation between the left, right and the oral branches 16, 18, 20 such that generally three points of contact, between the sensor body 4 and the face of the patient, are generally achieved and such contact assists with providing a stable supporting structure which generally prevents or minimizes rocking, rolling, pivoting, wobbling and/or other undesired motion of the temperature sensing device 2. It is to be appreciated that if the temperature sensing device 2 is used in combination with a cannula, the cannula can also have a similar shape, e.g., also have a similar or an identical upward acute angle B and a similar or an identical rearward acute angle C.

Once properly positioned on the face of a patient, the left and the right branches 16, 18 each extend in a lateral direction, above the upper lip but below the nasal septum of the nose of the patient, generally toward the ears of the patient so that each of the first and the second nasal thermocouples 6, 8 is readily positionable generally adjacent and centered with respect to a respective nasal cavity of the patient. Similarly, but independently of the left and right branches 16, 18, the lower branch 20 curves or bends slightly so as to position, as effectively as possible, the oral third temperature sensor 10 in the most advantageous position for detecting the oral temperature changes due to the patient's oral airflow. By arranging the lower branch 20, independent of the left and the right branches 16, 18, it is generally assured that the oral third temperature sensor 10 does not contact the patient's skin or mouth and thereby adversely influence the response of that temperature sensor to the oral airflow of the patient.

Due to the curved configuration of the left and the right branches 16, 18 and the lower branch 20, the sensor body 4 generally contacts the upper lip of the patient at least three different spaced apart locations, e.g., at each of the three remote ends 22, 24, 28 of the sensor body 4. As a result of such arrangement and contact between the sensor body 4 and the face of the patient, the sensor body 4, and thus the temperature sensing device 2, is essentially supported in a tripod-like fashion in a stabilized manner so that the sensor body 4 is essentially prevented from rocking, rolling, pivoting, wobbling and/or other undesired motion of the temperature sensing device 2 during a sleep diagnostic session. Since the temperature sensing device 2 contacts the patient's face generally at multiple contact points, the temperature sensing device 2 is less prone to rock from side to side or to roll up and down and thus less likely to contact the skin of the patient during a sleep diagnostic session.

Once positioned on a patient's face, the temperature sensing device 2 rests across the patient's upper lip and the leads 12, 14 each respectively extends across the patient's face and typically over and behind one of the ears of the patient. The leads 12, 14 may then be secured and/or adjusted, in a conventional manner, so that the temperature sensing device 2 will remain secured to the upper lip of a patient, firmly in place.

The first, the second and the third temperature sensors 6, 8, 10 and their respective circuits and the wire leads 12, 14, shown in FIG. 5, may be joined in any manner known in the art, for example, by soldering, crimping, taping, brazing or welding and may, if desired, to protect and insulate these joints and connections from the external environment.

The first and the second leads 12, 14 are connected to the respective circuit junctions of the nasal and oral circuits (only generally shown in FIG. 6) and can be coupled to desired circuitry for sending the resistivity change to a conditioning circuit, which is generally known in the art. As such, a further discussion concerning the same is not provided. The respiratory airflow detection circuit determines, in a conventional manner, the change in temperature across the thermocouples.

As shown in FIGS. 1-4 and 7 and 8, either a single substantially flat, generally rectangular, elongate surface or a pair of spaced apart elongate planar surfaces 30 and/or 32 may be affixed to the left and the right branches 16, 18 of the sensor body 4 in order to further assist with preventing or minimizing rocking, rolling, pivoting, wobbling and/or other undesired motion of the temperature sensing device 2. These planar surfaces 30, 32 provided a flat, as opposed to a rounded, support surface for the sensor body 4, and thus further assist with minimizing or preventing undesired rocking, rolling, pivoting, wobbling and/or other motion of the temperature sensing device 2 during a sleep diagnostic session. It is to be appreciated that the pair of spaced apart elongate planar surfaces 30 and/or 32 may be eliminated from the embodiment of FIGS. 1-4 and 7 and 8 without departing from the spirit of the present invention.

With reference to FIG. 9, another embodiment of the sensor body 4 is shown. According to this embodiment, the sensor body 4 generally defines a plane. That is, all three independent branches 16, 18, 20, which each extend from the central hub 26 of the sensor body 4 to a remote free end thereof 22, 24, 28, and only have curvature in one plane. That is, the sensor body 4 generally does not have any upward acute angle B but only has a rearward acute angle C. As a result of this curvature, each of the nasal thermocouples 6, 8 of the temperature sensing device 2 are still normally directly aligned in the flow path of the nasal airflow flowing through the nares of the patient while the lower branch 20 is independently still advantageously positioned for detecting the oral temperature changes due to the patient's oral airflow.

It is to be appreciated that not all the branches 16, 18, 20 necessarily have the same length. For example, the lower branch 20 is normally shorter than either the left or the right branches 16, 18 so that the lower branch 20 does not extend below the upper lip of the patient and thereby possibly position the oral sensor 10 below the oral airflow of the patient.

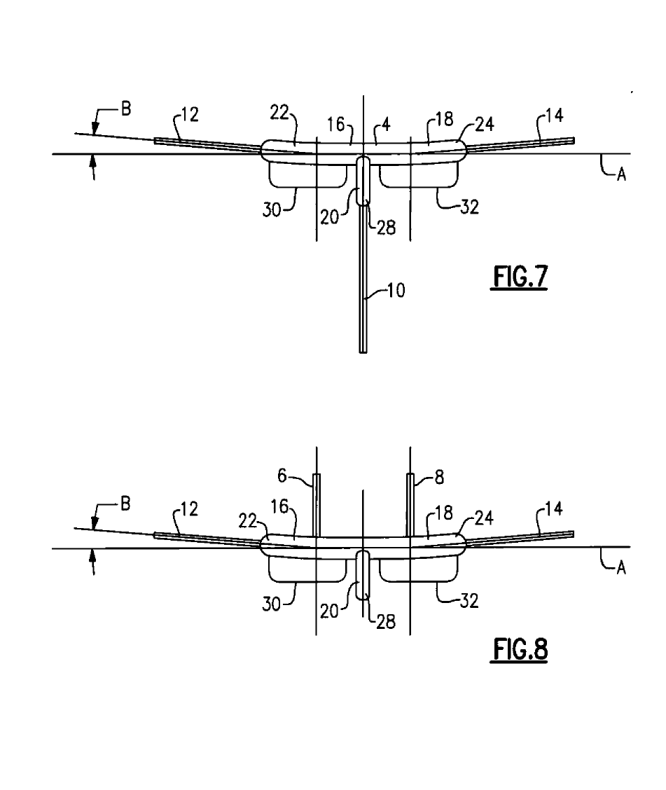

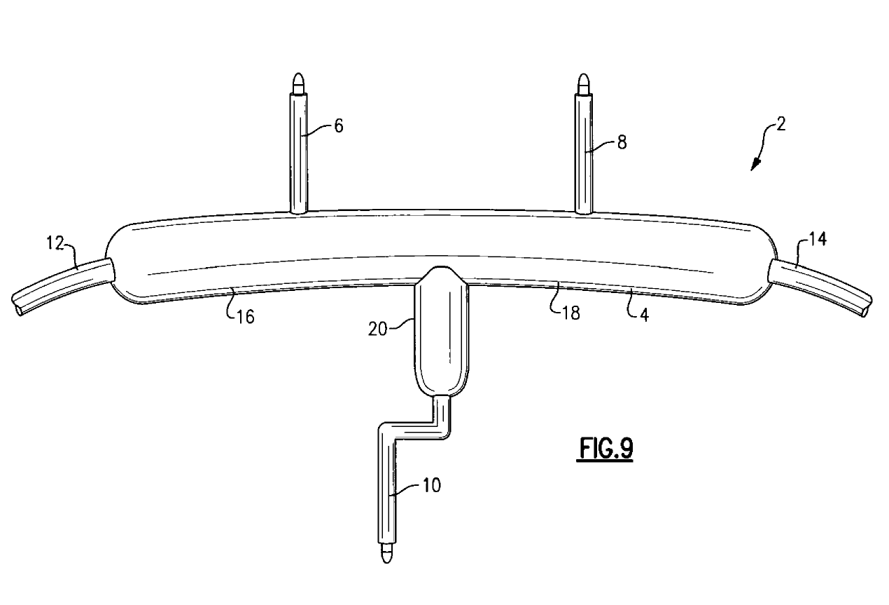

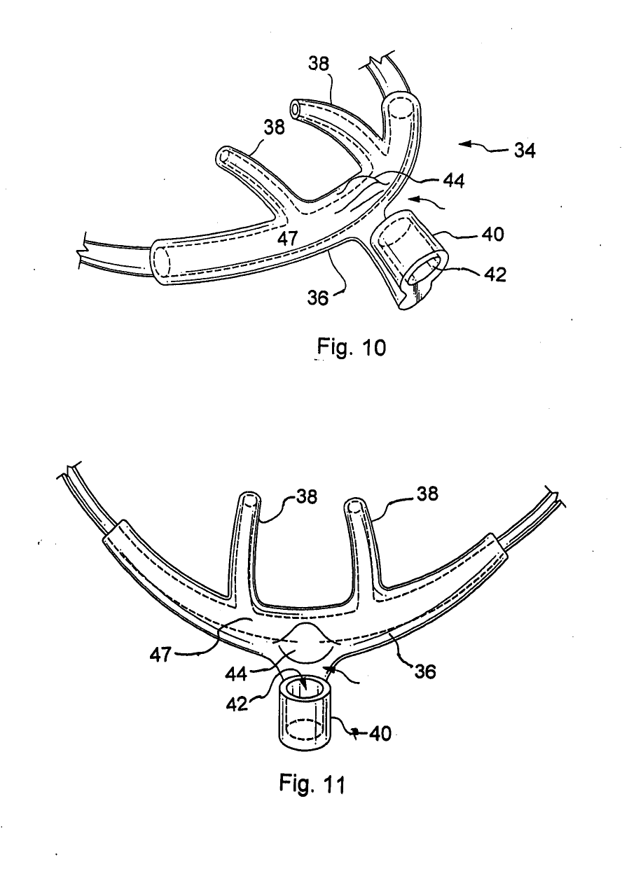

With reference now to FIGS. 10, 11 and 12, details of a suitable cannula 34 for use in the presently described system in conjunction with the above described temperature sensor device 2 and the circuit as shown in FIG. 6, for example, will now be described. The cannula 34 includes a main cannula body 36 which is hollow and undivided (i.e., an internal chamber 47 of the cannula body 36 does not include an internal dividing septum therein) and has first and second ends defining respective openings through which air and/or gas may be delivered to or received from a pair of nasal prongs 38, as are well known in the art, for receiving exhalation gases and/or supplying oxygen to the patient. The cannula 34 of this embodiment is further provided with an integral retaining mechanism, such as a holster 40, for example, which is integrally connected or formed with the body 36 of the cannula 34 and is provided with a sensor passage 42. The sensor passage 42 may be of any desired shape and, it is to be appreciated, does not have to be entirely enclosed, i.e., the sensor passage may be formed as a cylinder, for example, having a longitudinal slit S (see FIG. 13) which allows the size or internal dimension of the passage 42 to increase slightly as the third branch 20, having a diameter somewhat larger than the diameter of the passage 42, is received and retained therein by a frictional coupling or fit. Such frictional fit allows the temperature sensor device 2 to be securely and properly retained in position, during use, for sensing air flow from the patient, but also facilitates relatively easy removal thereof following the sleep diagnostic session.

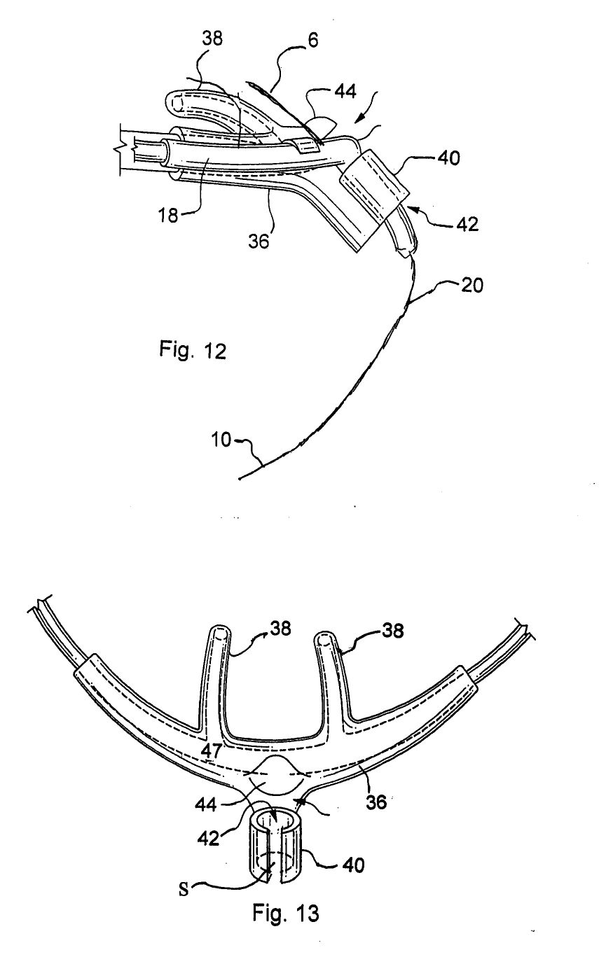

During assembly, a free end of the oral temperature sensor 10 of the temperature sensor device 2 is first inserted into a top opening of the sensor passage 42 and extends out through the opposed opening of the passage 42. As this occurs, the leading end of the lower branch 20 of the temperature sensor device 2 is generally forced into the top opening of the sensor passage 42. Due to the lower branch 20 of the temperature sensor device 2 preferably having a somewhat slightly larger diameter than the passage 42, as the leading end of the lower branch 20 passes through the passage 42 and projects out through a bottom of the passage 42 (see FIG. 12), the holster 40 will captively and securely retain the temperature sensor device 2 in position. The leading end of the lower branch 20 of the temperature sensor device 2 is preferably tapered, chamfered or rounded to facilitate insertion thereof into the passage 42. The lower branch 20 is normally forced into the passage 42 until the left and the right branches 16, 18, of the pressure sensing device 2, abut against the top surface of the sensor passage 42 and such abutment prevents further insertion of the lower branch 20 into the passage 42. As a result of such engagement, the temperature sensing device 2 is securely received and retained in a desired position by the holster 40. If desired, a central portion of the body 36 of the cannula 34, adjacent the holster 40, may include a retaining feature 44, e.g., a protrusion, a tab, a receiving space or notch, for example, which engages with a portion of the central hub 26, opposite the lower branch 20, to the assist with securely retaining the pressure sensing device 2 correctly positioned within the holster 40. The retaining feature 44 receives, engages and retains the central hub 26 but is sufficiently flexible to permit insertion and removal of the pressure sensing device 2 into and out of the passage 42 of the holster 40, as desired.

Once the temperature sensing device 2, as shown in FIG. 1-5 or 9 for example, is inserted into the sensor passage 42, and the combination is then placed on and supported between the nose and the upper lip of a patient for use in a sleep diagnostic session, each one of the temperature sensors 6, 8 or 10 may then be independently manipulated, if necessary, in order to appropriate position, align and/or curve the free ends of each of the temperature sensors 6, 8, 10 so as to facilitate the most reliable data collection position, as previously described.

With reference now to FIG. 14, a further embodiment of a divided cannula 34, for use in supporting the temperature sensor device 2 according to the present invention, will now be briefly described. As with the previous embodiments of FIGS. 10-13, this embodiment also includes a holster 40, an associated stop feature 44, a cannula body 36 defining an internal flow chamber 47 and a pair of nasal prongs 38. In addition, this embodiment further includes an internal septum 48 which is located within the cannula body 36 and divides the internal flow chamber into two completely separate supply/sensing flow paths, namely, a first flow path comprising one of the nasal prongs 38, a first section 47′ of the internal flow chamber and one of the supply/sensing flow tubes 50, 52 and a second flow path comprising the other one of the nasal prongs 38, a second section 47″ of the internal flow chamber and the other of the gas supply/sensing flow tubes 52, 50. As a result of this, each one of the separate supply/sensing flow paths can provide a separate and distinct function such as sense oral/nasal thermal airflow changes, supply oxygen, withdraw a CO₂ sample, detect changes in pressure, etc.

With reference now to FIG. 15, a further embodiment of an undivided cannula 34, for use in supporting the temperature sensor device 2 according to the present invention, will now be briefly described. As with the previous embodiments, this embodiment also includes a holster 40, an associated stop feature 44, a cannula body 36 defining an internal flow chamber 47 and a pair of nasal prongs 38 which each communicate with the internal flow chamber. In addition, the cannula 34 is also provided with an oral airflow pressure sensing prong 54 which communicates with the internal flow chamber 47 of the cannula body 36 as do the nasal prongs 38. The oral prong 54 may be either substantially centered relative to a centerline L of the cannula body 36 and the nasal prongs 38, or slightly offset to either side thereof. In order to ensure that the oral temperature sensor 10 is not blocked or obstructed in any manner by the supported temperature sensor device 2, preferably the holster 40 and the stop feature 44 (and the temperature sensing device 2 once attached thereto) are offset to one side of the cannula centerline L while the oral tube 54 is offset to the opposite side of centerline L. Such offset of these elements ensures that when the lower branch 20 of the temperature sensing device 2 is inserted into the holster 40, the oral temperature sensor 10 extends along the side of the oral prong 54, a free end of the oral temperature sensor 10 can be directly aligned adjacent the patient's oral airflow without being blocked or otherwise obstructed by the pressure sensing prong 54, or vice versa.

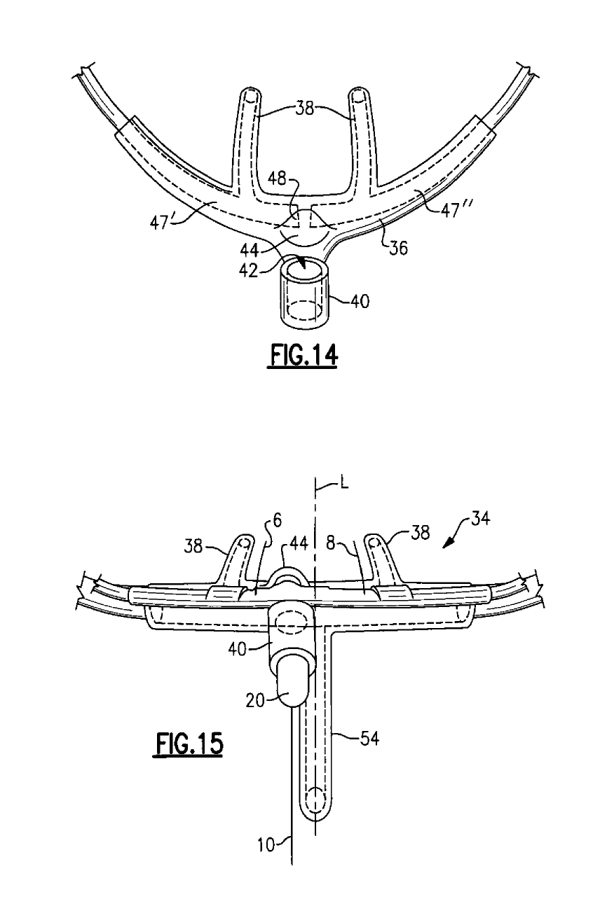

With reference now to FIG. 16, a still further embodiment of a divided cannula 34, for use in supporting the temperature sensor device 2 according to the present invention, will now be briefly described. As with the previous embodiment of FIG. 15, this embodiment also includes a holster 40, an associated stop feature 44, a cannula body 36 defining an internal flow chamber 47 and a pair of nasal prongs 38. This embodiment further includes a single oral airflow pressure sensing prong 54 which communicates with the cannula body 36 of the cannula 34. As with the previous embodiment of FIG. 14, this embodiment also includes an internal septum 48 which is located so as to divide the internal flow chamber 47 into two completely separate supply/sensing flow paths, namely, a first flow path comprising one of the nasal prongs 38, a first section 47′ of the internal flow chamber and one of the supply/sensing flow tubes 50, 52 and a second flow path comprising the other one of the nasal prongs 38, a second section 47″ of the internal flow chamber, the single oral airflow pressure sensing prong 54 and the other of the supply/sensing flow tubes 52, 50. As a result of this, each one of the separate gas supply/sensing flow paths can provide a separate and distinct function such as detecting or sensing oral/nasal thermal airflow changes, supplying oxygen, withdrawing a CO₂ sample, detecting changes in pressure, etc. As with the previous embodiment, the oral tube 54 may be either substantially centered relative to a centerline L of the cannula body 36 and the nasal prongs 38, or slightly offset to one side or the other thereof. In order to ensure that the temperature sensor 10 is not, in any manner, blocked or obstructed by for the supported temperature sensor device, preferably the holster 40 and the stop feature 44 (and the temperature sensing device 2 once attached thereto) are offset to one side of the cannula centerline L while the oral tube 54 is offset to an opposite side of centerline L. Such offset of these elements ensures that when the lower branch 20 of the temperature sensing device 2 is inserted into the holster 40, the lower branch 20 extends along the side of the oral tube 54 and the oral temperature sensor 10 can be directly aligned adjacent the patient's oral airflow without being blocked or otherwise obstructed by the pressure sensing prong 54, or vice versa.

With reference now to FIG. 17, a still further embodiment of an undivided cannula 34, for use in supporting the temperature sensor device 2 according to the present invention, will now be briefly described. As with the previous embodiments, this embodiment also includes a holster 40, an associated stop feature 44, a cannula body 36 defining an internal flow chamber 47 and a pair of nasal prongs 38 which each communicate with the internal flow chamber. In addition, this embodiment includes a pair of oral airflow pressure sensing prongs 54, 54′ only one of which, i.e., the sensing prong 54, normally communicates, in addition to one of the nasal prongs 38, with the internal flow chamber 47 of the cannula body 36 the other prong of which, i.e., the prong 54′, is sized for receiving and accommodating a malleable shape retaining member or a “dead soft” material (only diagrammatically shown) which enables the oral prong 54′ to be bent, shaped, molded or otherwise configured into a desired curvature or orientation for positioning a detection opening 64 of the oral prong 54 at a desired position relative to the mouth or the oral cavity of a patient for detecting or sensing breathing of the patient.

The holster 40 and the stop feature 44 are both generally substantially centered with respect to a centerline L of the cannula body 36 and the nasal prongs 38 while each of one of the oral tubes 54, 54′ is offset on either side of the centerline L of the cannula body 36. In order to ensure that the oral temperature sensor 10 is not, in any manner, blocked or obstructed by the supported temperature sensor device 2, the pair of the oral tubes 54, 54′ are sufficiently spaced apart from one another so that the oral temperature sensor 10 may be located therebetween. Such arrangement of these elements ensures that when the lower branch 20 of the temperature sensing device 2 is inserted into the holster 40, the lower branch 20 extends along the side and between both of the oral tubes 54, 54′ so that the oral temperature sensor 10 can be directly aligned adjacent the patient's oral airflow without being blocked or otherwise obstructed by either one of the pressure sensing prongs 54, 54′, or vice versa.

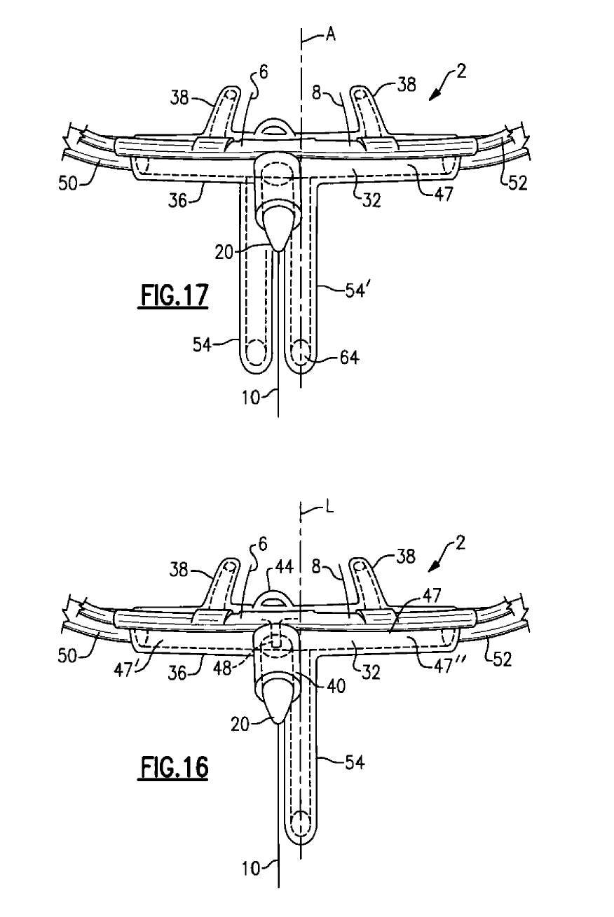

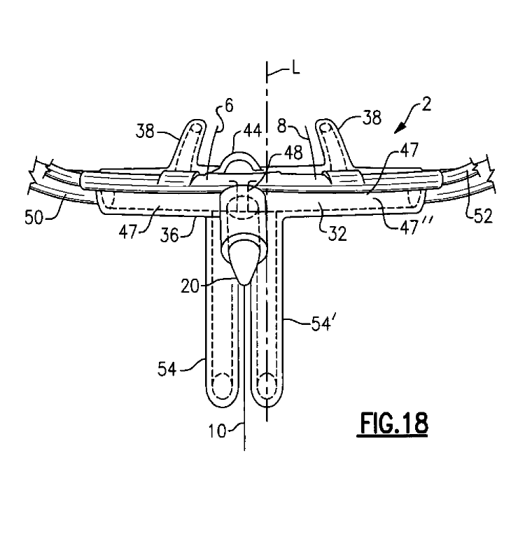

With reference now to FIG. 18, this figure is a diagrammatic illustration of a divided cannula having dual oral sensing oral prongs for use in supporting the temperature sensor device 2, according to the present invention. As with the previous embodiments, this embodiment also includes a holster 40, an associated stop feature 44, a cannula body 36 defining an internal flow chamber 47 and a pair of nasal prongs 38. The embodiment further includes a pair of oral airflow pressure sensing prongs 54, 54′ which each communicate with the internal flow chamber 47 of the cannula body 36. According to this embodiment, an internal septum 48 is located within an internal chamber 47 of the cannula body 36, at a location between the pair of oral prongs 54, 54′, so as to divide the internal flow chamber 47 of the cannula 34 into two completely separate gas supply/sensing flow paths, namely, first and second sections 47′, 47″ of the internal flow chamber. That is, a first one of the nasal prongs 38 and a first one of the oral airflow pressure sensing prongs 54 both communicate with one another and with a first one of the gas supply/sensing flow tubes 52, 50 via the first section 47′ of the internal flow chamber to form a first separate flow path while a second one of the nasal prongs 38 and a second one of the oral airflow pressure sensing prongs 54′ both communicate with one another and with the other one of the gas supply/sensing flow tubes 50, 52 via the second section 47″ of the internal flow chamber so as to form a second separate flow path. As a result of this, each one of the separate first and second gas supply/sensing flow paths can provide a separate and distinct function such as detecting or sensing oral/nasal thermal airflow changes, supplying oxygen, withdrawing a CO₂ sample, detecting changes in pressure, etc.

As with the previous embodiment of FIG. 17, the holster 40 and the stop feature 44 are generally substantially centered relative to a centerline L of the cannula body 36 and the nasal prongs 38 while each of one of the oral tubes 54, 54′ is offset on either side of the centerline L of the cannula body 36. In order to ensure that the oral temperature sensor 10 is not blocked or obstructed, in any manner, by the supported temperature sensor device 2, the pair of the oral tubes 54, 54′ are sufficiently spaced apart from one another so that the oral temperature sensor 10 may be located therebetween. Such arrangement of these elements ensures that when the lower branch 20 of the temperature sensing device 2 is inserted into the holster 40, the oral temperature sensor 10 extends along the side and between both of the oral tubes 54, 54′ and the oral temperature sensor 10 and can be directly aligned adjacent the patient's oral airflow without being blocked or otherwise obstructed by either one of the pressure sensing prongs 54, 54′, or vice versa.

Turning now to FIG. 19, this figure is a diagrammatic illustration of a variation of a divided cannula having dual oral sensing oral prongs for use in supporting the temperature sensor device 2, according to the present invention. As with the previous embodiments, this embodiment also includes a holster 40, an associated stop feature 44, a cannula body 36 and a pair of nasal prongs 38, and a pair of oral airflow pressure sensing prongs 54, 54′ which each communicate with the cannula body 36. However, according to this embodiment, the cannula body 36 defines two completely separate internal flow chambers 47 and 49 and the pair of nasal prongs 38 both communicate with the first internal flow chamber 47 while the pair of oral airflow pressure sensing prongs 54, 54′ both communicate with the second internal flow chamber 49. An internal septum 48 is located within the first internal chamber 47 of the cannula body 36, at a located between the pair of nasal prongs 38, so as to divide the first internal flow chamber 47 of the cannula 34 into two completely separate gas supply/sensing flow paths, namely, first and second sections 47′, 47″ of the first internal flow chamber. That is, a first one of the nasal prongs 38 communicates with one a first one of the gas supply/sensing flow tubes 52, 50 via the first section 47′ of the first internal flow chamber to form a first separate flow path while a second one of the nasal prongs 38 communicates with one another one of the gas supply/sensing flow tubes 52, 50 via the second section 47″ of the first internal flow chamber so as to form a second separate flow path. In addition, the pair of oral airflow pressure sensing prongs 54, 54′ both communicate with the second internal flow chamber 49 of the cannula 34 to form a third completely separate gas supply/sensing flow path, namely, the pair of oral airflow pressure sensing prongs 54, 54′ both communicate with one another and with the gas supply/sensing flow tubes 56, 58 via the second internal flow chamber 49 so as to form a third separate flow path.

As with the previous embodiment of FIGS. 17, 18 and 19, the holster 40 and the stop feature 44 are generally substantially centered relative to a centerline L of the cannula body 36 and the nasal prongs 38 while each of one of the oral tubes 54, 54′ is offset on either side of the centerline L of the cannula body 36. In order to ensure that the oral temperature sensor 10 is not blocked or obstructed, in any manner, by the supported temperature sensor device 2, the pair of the oral tubes 54, 54′ are sufficiently spaced apart from one another so that the oral temperature sensor 10 may be located therebetween. Such arrangement of these elements ensures that when the lower branch 20 of the temperature sensing device 2 is inserted into the holster 40, the lower branch 20 extends along the side and between both of the oral tubes 54, 54′ and the oral temperature sensor 10 and can be directly aligned adjacent the patient's oral airflow without being blocked or otherwise obstructed by either one of the pressure sensing prongs 54, 54′, or vice versa.

Turning now to FIG. 20, this figure is a diagrammatic illustration of a combined multifunction dual divided cannula for use in supporting the temperature sensor device 2, according to the resent invention. As with the previous embodiment of FIG. 19, this embodiment also includes a holster, an associated stop feature 44, a cannula body 36, a pair of nasal prongs 38, and a pair of oral airflow pressure sensing prongs 54, 54′ with each nasal and oral prong communicating with the cannula body 36. However, according to this embodiment, the cannula body 36 defines two completely separate internal flow chambers 47 and 49 and the pair of nasal prongs 38 both communicate with the first internal flow chamber 47 while the pair of oral airflow pressure sensing prongs 54, 54′ both communicate with the second internal flow chamber 49. A first internal septum 48 is located within the first internal flow chamber 47 of the cannula body 36, at a location between the pair of nasal prongs 38, so as to divide the first internal flow chamber 47 of the cannula 34 into first and second sections 47′, 47″ of the first internal flow chamber 47. That is, a first one of the nasal prongs 38 communicates with a first one of the gas supply/sensing flow tubes 50, 52 via the first section 47′ of the first internal flow chamber 47 to form a first separate flow path while a second one of the nasal prongs communicates with another one of the gas supply/sensing flow tubes 52, 50 via the second section 47″ of the first internal flow chamber so as to form a second separate flow path.

In addition, a second internal septum 48′ is located within the second internal chamber 49 of the cannula body 36, at a location between the pair of oral airflow pressure sensing prongs 54, 54′, so as to divide the second internal flow chamber 49 into two completely separate gas supply/sensing flow paths, namely, first and second sections 49′, 49″ of the second internal flow chamber 49. That is, a first one 54 of the pair of oral airflow pressure sensing prongs 54, 54′ communicates with a third one of the gas supply/sensing flow tubes 56, 58 via the first section 49′ of the second internal flow chamber 49 to form a third separate flow path while a second one 54′ of the pair of oral airflow pressure sensing prongs 54, 54′ communicates with another one of the gas supply/sensing flow tubes 56, 58 via the second section 49″ of the second internal flow chamber 49 so as to form a fourth separate flow path. As a result of this, each one of the four completely separate gas supply/sensing flow paths can provide a separate and distinct function such as detecting or sensing oral/nasal thermal airflow changes, supplying oxygen, withdrawing a CO₂ sample, supplying O₂, detecting changes in pressure, etc.

As with the previous embodiment of FIGS. 17, 18 and 19, the holster 40 and the stop feature 44 are generally substantially centered relative to a centerline L of the cannula body 36 and the nasal prongs 38 while each of one of the oral tubes 54 is offset on either side of the centerline L of the cannula body 36. In order to ensure that the oral temperature sensor 10 is not, in any manner, blocked or obstructed by the supported temperature sensor device 2, the pair of the oral tubes 54 are sufficiently spaced apart from one another so that the oral temperature sensor 10 may be located therebetween. Such arrangement of these elements ensures that when the lower branch 20 of the temperature sensing device 2 is inserted into the holster 40, the lower branch 20 extends along the side and between both of the oral tubes 54 and the oral temperature sensor 10 and can be directly aligned adjacent the patient's oral airflow without being blocked or otherwise obstructed by either one of the oral prongs 54, or vice versa.

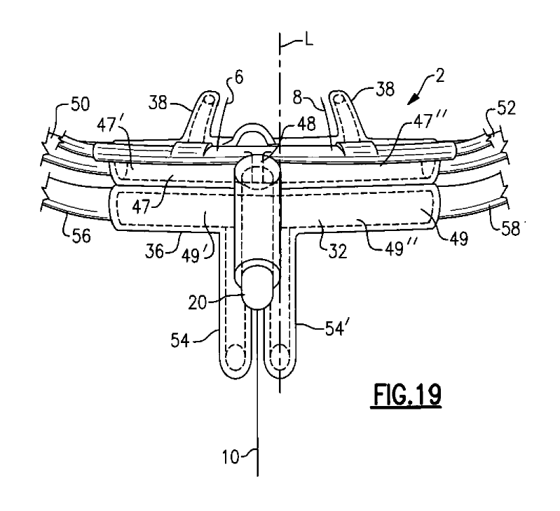

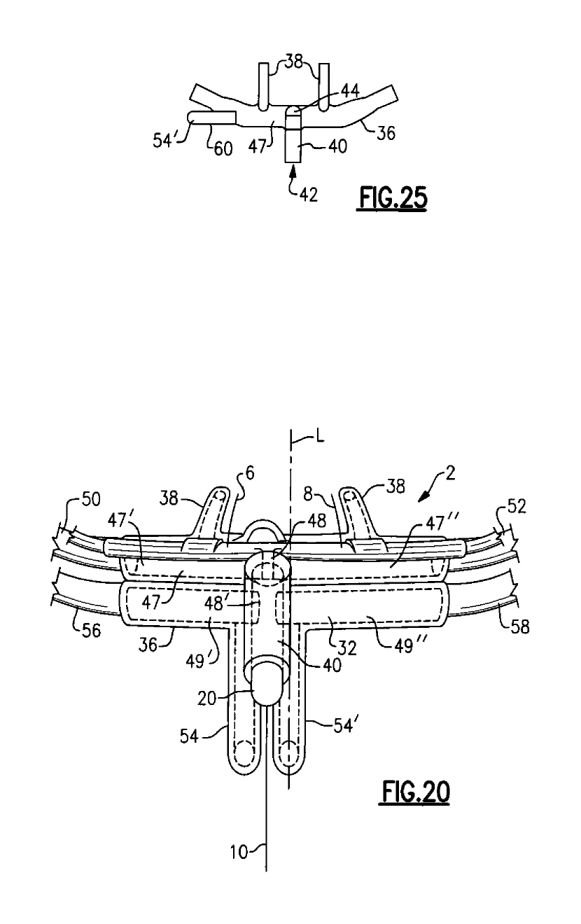

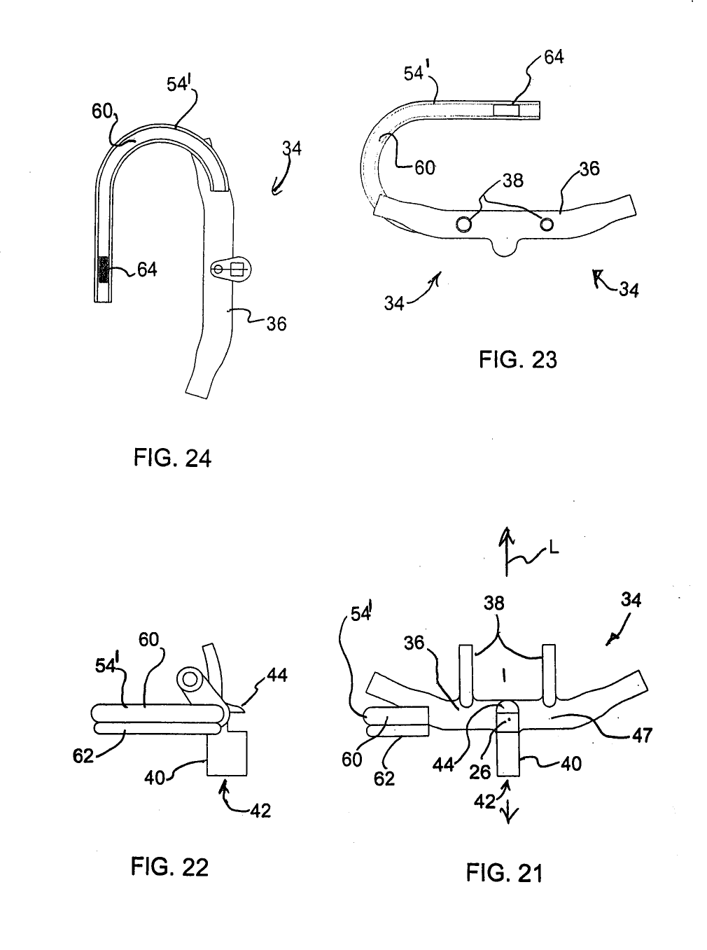

With reference now to FIGS. 21 through 24, another embodiment of an undivided cannula having an oral prong with dual passageways, for use with the temperature sensor according to the present invention, will now be discussed. To facilitate a more complete understanding of this embodiment, without repeating the above description, similar or identical elements are provided with the same reference numerals in the following description and the referenced drawings.

As with the previous embodiments, the cannula 34 includes a main cannula body 36 which is hollow and undivided and has first and second ends defining respective openings through which air and/or gas may be delivered to or information may be received from a pair of nasal prongs 38. A central front section of the cannula 34 is provided with an integral holster 40 which is integrally connected or formed with the body 36 of the cannula 34 and the holster 40 is provided with a sensor passage 42, as discussed above. The central portion of the body 36 of the cannula 34, adjacent the holster 40, typically includes a retaining feature 44, e.g., typically a sufficiently flexible protrusion, tab, receiving space or notch for example, which engages with a portion of a central hub 26 to the assist with securely retaining the pressure sensing device 2 within the holster 40 once properly installed.

As with the previous embodiments, this embodiment also includes an oral airflow pressure sensing prong 54′ which communicates, along with the nasal prongs 38, with the internal flow chamber 47 of the cannula body 36. However, according to this embodiment, the oral prong 54′ is “bow shaped” or initially projects away and substantially normal to the centerline L of the cannula body 36 (see FIGS. 23 and 24), rather than extending therealong, and the oral airflow pressure sensing prong 54′ thereafter eventually curves and bends back 180 degrees toward the centerline L of the cannula body 36. In addition, according to this embodiment, the bow shaped oral prong 54′ is a dual passageway prong which defines a first passage 60, for detecting oral breathing data, detecting or sensing oral/nasal thermal airflow changes, detecting changes in pressure, withdrawing a CO₂ sample, supplying O₂, etc., and also defines a second passage 62 for receiving a malleable shape retaining member or a “dead soft” material (only diagrammatically shown) which enables the bow shaped oral prong 54′ to be bent, shaped, molded or otherwise configured into a desired curvature or orientation for positioning a detection opening 64 of the bow shaped oral prong 54′ at a desired position relative to the mouth or the oral cavity of a patient for detecting or sensing breathing of the patient.

As shown in the drawings, a first end of the first passage 60 of the bow shaped oral prong 54′ communicates with the internal flow chamber 47 of the cannula body 36 while a second free end of the first passage 60 has the detection opening 64 formed therein which can be located adjacent the mouth of the patient, during use, for detecting oral breathing information, detecting or sensing oral/nasal thermal airflow changes, detecting changes in pressure, withdrawing a CO₂ sample, supplying O₂, etc. The detection opening 64 may possibly be in the end face of the bow shaped oral prong 54′, which operates adequately but is not preferred since such opening generally extends parallel to the centerline L of the cannula body 36, rather than normal thereto. More preferably the detection opening 64 is an (rectangular, oval, circular, etc.) aperture or cutout formed in a sidewall of the bow shaped oral prong 54′ which directly faces and communicates with the inspiration and expiration oral airflow of a patient during use. This arrangement completely spaces the detection opening 64, of the bow shaped oral prong 54′, away from the oral temperature sensor 10 so that both the oral temperature sensor 10 and the detection opening 64 are not, in any manner, blocked or obstructed by one another and this tends to lead to more precise data collection during a sleep diagnostic session.

As shown in FIGS. 23 and 24, the bow shaped oral prong 54′ bends approximately 180 degrees so that the free end of the bow shaped oral prong 54′ generally extends substantially parallel to a central portion of the cannula body 36. Generally, an internal diameter of the first passage 60 is between 0.05 and 0.125 inches, preferably about 0.075 inches while an internal diameter of the second passage 62 is between 0.04 and 0.10 inches, preferably about 0.06 inches.

Turning now to FIG. 25, another variation of the temperature sensor embodiment of FIGS. 21 through 24 will now be briefly discussed. The major difference between this embodiment and the embodiment of FIGS. 21 through 24, is that the oral airflow pressure sensing prong 54′ only defines the first passage 60, for detecting oral breathing data, detecting or sensing oral/nasal thermal airflow changes, detecting changes in pressure, withdrawing a CO₂ sample, supplying O₂, etc., but does not include a second passage for receiving a malleable shape retaining member or a “dead soft” material which enables the bow shaped oral prong 54′ to be bent, shaped, molded or otherwise configured into a desired curvature or orientation. In all other respects, this embodiment is the same as the embodiment described with respect to FIGS. 21-24 and thus a further description concerning the same is not provided.

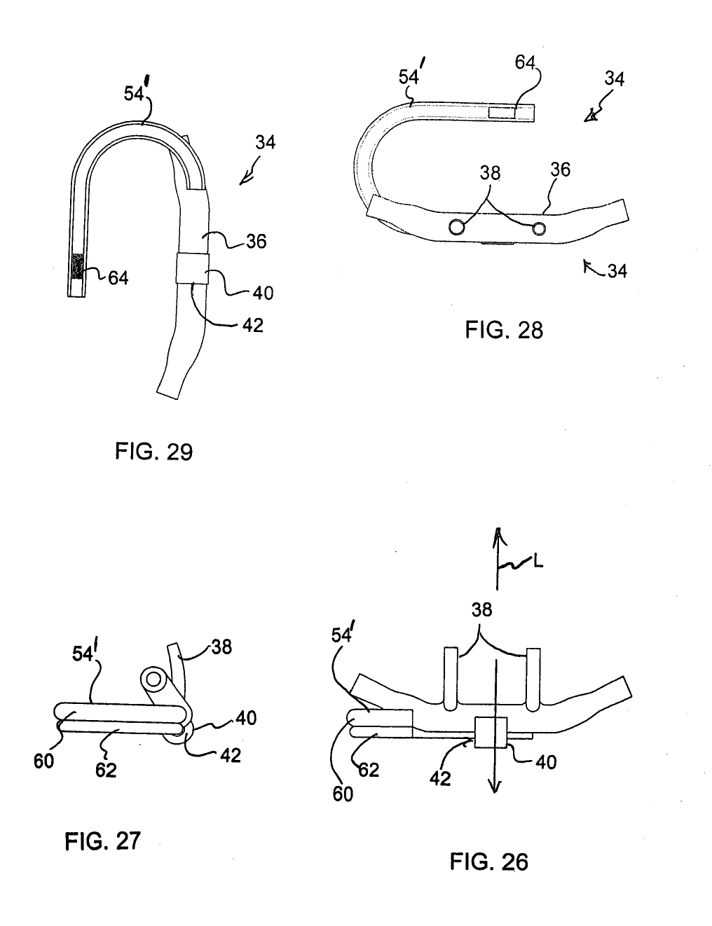

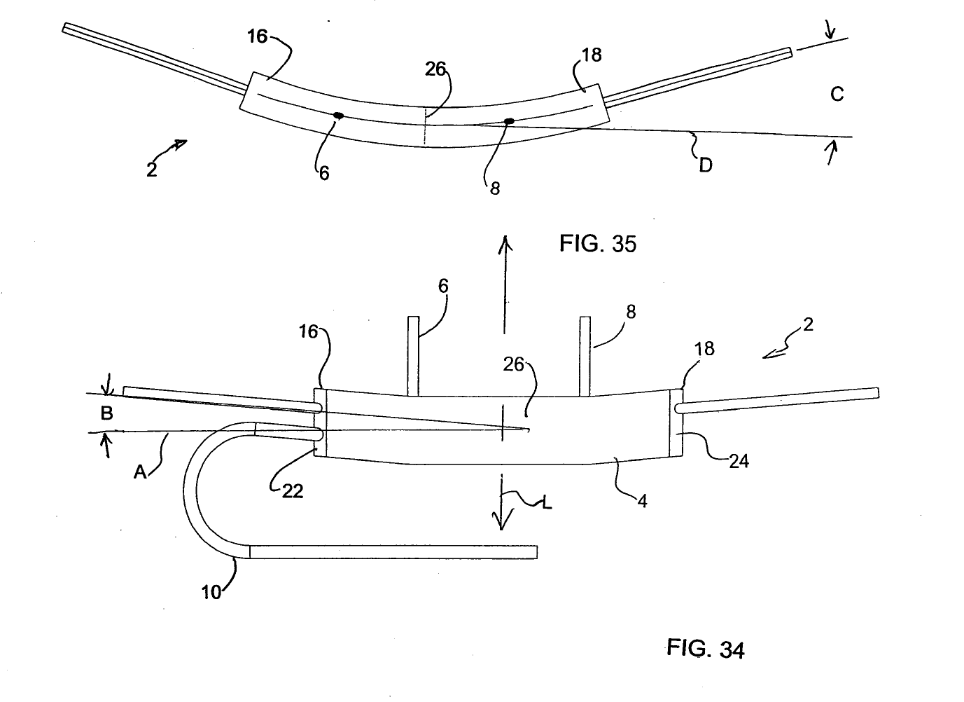

With reference now to FIGS. 26-29, a slight variation of the embodiment of FIGS. 21 through 24, for use with the temperature sensor according to the present invention, will now be discussed. The major difference between this embodiment and the two immediately preceding embodiments, is the arrangement of the holster 40. According to this embodiment, in order to facilitate attachment of a desired temperature sensing device 2 (such as shown in FIGS. 34 and 35), to the cannula body 36 adjacent the first and the second nares or nasal prongs 38, the cannula body 36 is provided with a single centrally located holster 40, or some other mechanism, which facilitates receiving and positioning the temperature sensing device 2 at a desired location precisely between the first and the second nares or nasal prongs 38 of the cannula body 36. The holster 40 has a sensor passage 42 formed therein which extends through and along a length of the holster 40, substantially parallel to the cannula body 36, to facilitate receiving and supporting the desired temperature sensor device 2. The holster 40 may be cylindrical in shape and have a length of between about 0.4 and about 0.5 inches, contain an opening or through bore having a diameter of between about 0.08 and about 0.10 inches and have an exterior dimension of between about 0.15 and about 0.19 inches. It is to be appreciated that the single holster 40 may have an elongate cut, slot or opening formed therein which extends along the entire axial length of the side wall of the holster 40 (not shown but similar to slot S shown in FIG. 13) to facilitate expanding the size or diameter of the holster 40 somewhat to allow accommodation of different diameter and/or sized temperature sensor device 2. The cannula body 36 also includes a bow shaped oral prong 54′ with a detection opening 64 provided in the free end thereof, as discussed above.

According to this embodiment, following insertion and engagement of the temperature sensor device 2 with the single holster 40, the first and the second temperature sensors 6, 8 are each correctly located and positioned adjacent one of the first and the second nares or nasal prongs 38 of the cannula body 36 so that the airflow being inspired and expired by the patient's nostrils will contact the respective first or second temperature sensor 6, 8 and facilitate detection of the temperature of the inspired and expired airflow while the third temperature sensor 10 is located for desired positioning adjacent the mouth of the patient so that the airflow being orally inspired and expired by the patient will contact the third temperature sensor 10 and facilitate detection of the temperature of the orally inspired and expired airflow.

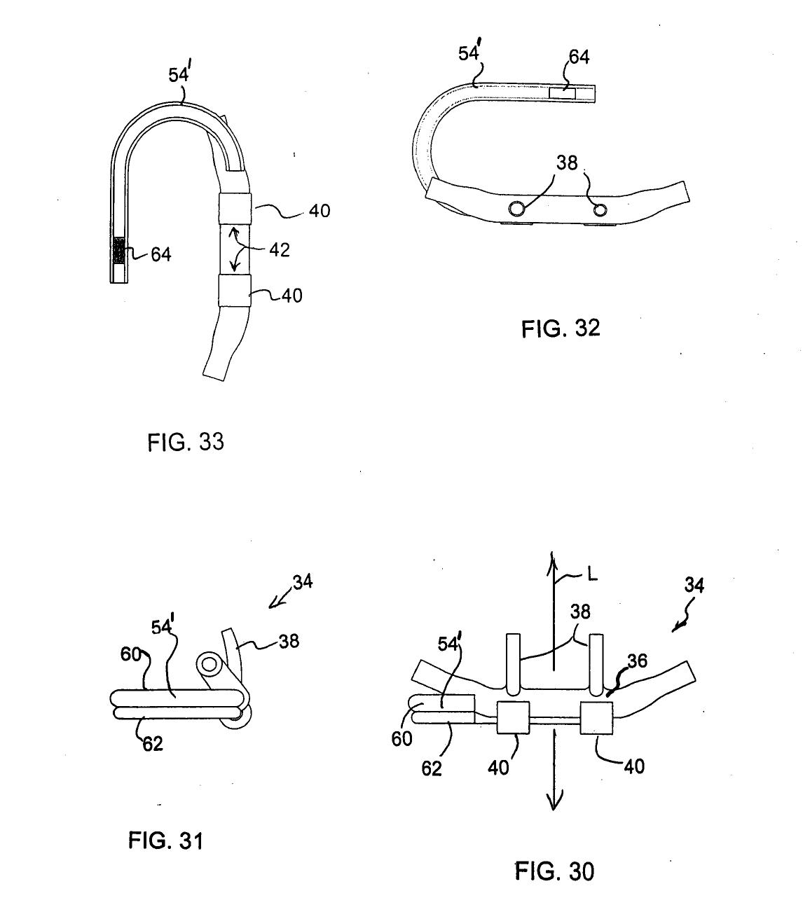

With reference now to FIGS. 30-33, a slight variation of the embodiment shown and described with respect to FIGS. 26-29 will now be discussed. The major difference between this embodiment and the immediately preceding embodiment of FIGS. 26-29, is the arrangement of the holster 40. According to this embodiment, in order to facilitate attachment of a desired temperature sensing device 2 to the cannula body 36, adjacent the first and the second nares or nasal prongs 38, the cannula body 36 is provided with a pair of holsters 40 which are spaced apart from one another but generally aligned with one another so as to facilitate receiving and positioning the temperature sensing device 2 at a desired location between the first and the second nares or nasal prongs 38, 38 of the cannula body 36. Each of the aligned holsters 40 has a sensor passage 42 formed therein which extends either partially or completely through the respective holsters 40, generally parallel to the cannula body 36, to facilitate receiving and supporting the desired temperature sensor device 2 therein. Each holster 40 may be cylindrical in shape and have a length of between about 0.4 and about 0.5 inches, contain an opening or through bore having a diameter of between about 0.08 and about 0.10 inches and have an exterior dimension of between about 0.15 and about 0.19 inches. Each of the pair of holsters 40 may have an elongate cut, slot or opening formed therein and extending the entire axial length of the side wall of the holster 40 (not shown but similar to slot S shown in FIG. 13) to facilitate expanding of the diameter of one or both of the holsters 40 somewhat to allow accommodation of different diameter and/or sized temperature sensor devices 2.

Following insertion and engagement of the temperature sensor device 2 with the pair holsters 40, the first and the second temperature sensors 6, 8 are each correctly located and positioned adjacent one of the first and the second nares or nasal prongs 38, 38 of the cannula body 36 so that the airflow being inspired and expired, from the patient's nostrils, will contact the respective first or second temperature sensor 6, 8 and facilitate detection of the temperature of the inspired and expired airflow while the third temperature sensor 10 is located for desired positioning adjacent the mouth of the patient so that the airflow being orally inspired and expired by the patient will contact the third temperature sensor 10 and facilitate detection of the temperature of the orally inspired and expired airflow

With reference now to FIGS. 34 and 35, a variation of the temperature sensor of FIGS. 1-9, for use with according to the present invention, will now be discussed. The major difference between this embodiment and the embodiment of FIGS. 1-9, is that this embodiment is designed as a more durable temperature sensor device 2, following conventional cleaning and/or sterilization thereof, and the exit position of the third temperature sensor 10 is modified so as to extend from one of the opposed ends, rather than the center, of the sensor body 4.

As with the previous embodiment, the sensor body 4, the sensor body is shaped and curved in two planes so as to allow the sensor body 4 to rest securely and comfortably, on the upper lip of a patient, without the need to use adhesive tape in order to stabilize the sensor body 4 and prevent the sensor body from rolling, twisting, wobbling or the like. That is, the left and the right branches 16, 18 are molded and curved so as to form a slightly upward acute angle B with respect to a horizontal plane A passing horizontally through a central area of the sensor body 4, as shown in FIG. 34, such that the remote ends 22, 24 of the left and the right branches 16, 18 preferably curve and extend slightly upward generally toward the patient's ears, once the temperature sensing device 2 is installed above the upper lip of the patient. The left and the right branches 16, 18 are also molded and curved so as to form a rearward acute angle C, as shown in FIG. 35, with respect to a vertical plane D passing through the central hub 26 of the sensor body 4 such that the left and right branches 16, 18 also bend or extend rearward toward the patient's ears, once the temperature sensing device 2 is installed above the upper lip of the patient. In addition, a thickness and/or width of the sensor body 4 may be increased, over the dimensions shown in the embodiment of FIGS. 1-4, 7 and 8 to render the temperature sensor device 2 more durable and thus reusable.

As shown in FIGS. 34 and 35, the third temperature sensor 10 is generally “bow shaped” or initially projects from one end of the sensor body 4 away and substantially normal to the centerline L of the central hub 26 of the sensor body 4 and thereafter eventually curves or bends back toward the centerline L of the sensor body 4. Preferably a portion of the third temperature sensor 10, located adjacent the sensor body 36, is covered with a plastic overmolded material or some other protective barrier 66, which protects the third temperature sensor 10 and also provides additional rigidity thereto to assist with, when the temperature sensor device 2 is used in combination with a nasal cannula, either “feeding” and/or “threading” of a leading end of the third temperature sensor 10 through the passage(s) 42 of the holster(s) 40 or through the second passage 62 of the bow shaped oral prong 54′ so as to be captively retained by the cannula body 36 while still positioning the free end of the third temperature sensor 10 ata desired position relative to the mouth of the patient.

In addition, according to this embodiment, the temperature sensor 10 is preferably manufactured from a malleable shape retaining or “dead soft” material which enables the third temperature sensor 10 to be bent, shaped, molded or otherwise configured into a desired curvature or orientation for positioning a free end thereof at a desired detection position relative to the mouth or the oral cavity of a patient for detecting pressure and/or other breathing information of the patient. In all other respects, this embodiment is the same as the embodiment described with respect to FIGS. 1-4, 7 and 8 and thus a further description concerning the same is not provided.

Turning now to FIG. 36, another variation of the embodiment of the temperature sensor device 2 according to the present invention will now be described. The major difference between this embodiment and the embodiment of FIGS. 34 and 35 is that the temperature sensor device 2 is designed for an infant rather than for an adult and thus the sensor body 4 is only provided with a single temperature sensor 6, rather than first and second temperature sensors, which is generally located or positioned between the nostrils of the infant patient. It is also possible that the sensor body 4 may not be curved in either plane or may only be slightly curved in one plane, e.g., to allow the sensor body 4 to rest securely and comfortably, on the upper lip of the infant patient, without the need to use adhesive tape to stabilize the sensor body to prevent the sensor body 4 from rolling, twisting, wobbling or the like. In all other respects, this embodiment is the same as the embodiment described with respect to FIGS. 1-4, 7, 8, 34 and 35 and thus a further description concerning the same is not provided.

One aspect of the present invention is to sufficiently space the detecting surface of the temperature sensor 6, 8, 10 from the exterior surface of the cannula body 32, 36 of the cannula 34 so as to avoid any contact between those surfaces. It is to be appreciated that if the cannula 34, or any other surface, is located too close to or contacts the detecting surface of the temperature sensor 6, 8, 10, this can disrupt accurate temperature sensing by the respective temperature sensor. Preferably the detecting surface of the temperature sensor 6, 8, 10 is spaced from the exterior surface of the cannula 34 by a distance of between about 0.040 and 0.080 inches or so.

It is to be appreciated that the temperature sensor device 2, according to the present invention, can be used with sufficiently large cannulas that may be utilized with adults and also may be use with smaller cannulas that are specifically suited for use with smaller patients such as young adults, children and infants.

It is to be appreciated that the temperature sensor device 2, according to the present invention, can either be directly support on the face of a patient or, alternatively, the temperature sensor device 2 may be attached to and carried by a suitable cannula, whether or not the cannula is divided or undivided, and whether or not the cannula has one or more oral prongs. In addition, each one of the completely separate internal flow paths is suitable for one of: (1) supplying a treating gas to a patient, (2) withdrawing or sampling an exhalation gas(es) from the patient, (3) monitoring breathing characteristics of the patient, (4) detecting pressure fluctuations during breathing, etc.

It is to be appreciated that the temperature sensor device 2 may be designed as an inexpensive single use device which is typically only used for a single sleep diagnostic session and thereafter properly discarded. Alternatively, the temperature sensor device 2 may be a more expensive and durable device which is designed for multiple uses, e.g., it is designed to be used for many sleep diagnostic sessions following proper cleaning and/or sterilization after each use thereof. However, once the temperature sensor device 2 becomes defective or malfunctions for some reason, the temperature sensor device 2 is generally discarded and replaced with a new temperature sensor device 2.

The term retaining or “dead soft” material, as used within this application, is intended to mean a material which has substantially no structural memory of any previous shape, orientation, configuration or form which would cause the material to retain, return or spring back to such previous shape, orientation, configuration or form. A suitable example of the retaining or dead soft material, to be used as a shape retaining support in the manufactured cannula, is copper wire, either insulated or uninsulated, although other dead soft materials, for example, other metals or plastics materials, would also be suitable for use with the present invention. Copper is a highly malleable metal and generally retains whatever shape is imparted thereto at any particular time without reverting or returning back to any prior or previous shape. Copper is also a preferred dead soft material, over for example iron, steel or other ferromagnetic materials, due to the propensity of the nasal cannula to be used on a patient exposed to certain electromagnetic and magnetic environments and/or diagnosis procedures.

It is to be appreciated that while the “retaining mechanism” is generally described as a holster, as set forth above within this application, the term retaining mechanism, as utilized herein, is intended to mean, include and cover a variety of other arrangements, mechanisms, means, clamps, elements and/or features which generally facilitate releasable connection or attachment of the temperature sensing device to the main body of the cannula so the each of the temperature sensors is correctly positioned either adjacent or at least partially within either one of the nasal cavities or the oral cavity to sense breathing by the patient, while still permitting removal of the temperature sensing device following use thereof. An important aspect of the “retaining mechanism” is that it sufficiently retains the temperature sensing device is a correct sensing position while also facilitates removal thereof following use.

Since certain changes may be made in the above described temperature sensor, without departing from the spirit and scope of the invention herein involved, it is intended that all of the subject matter of the above description or shown in the accompanying drawings shall be interpreted merely as examples illustrating the inventive concept herein and shall not be construed as limiting the invention.

Wherefore, I/We claim:

1. A combined cannula and temperature sensing device for detecting breathing of a patient, the cannula comprising:
a cannula body defining at least one internal flow chamber therein;
at least one tube being connected to the cannula body and communicating with the internal flow chamber;
a pair of nasal prongs communicating with the internal flow chamber, and
the cannula body having retaining mechanism for releasably attaching the temperature sensing device to the cannula; and
the temperature sensing device comprising:
a sensor body;
at least one temperature sensor being supported by the sensor body, and the sensor body releasably engaging with the retaining mechanism of the cannula for positioning the at least one temperature sensor adjacent at least one of a nasal cavity and a mouth of the patient for detecting breathing of the patient.

2. The combined cannula and temperature sensing device according to claim 1, wherein the retaining mechanism comprises a passage and the sensor body has an interference fit with the passage for releasably connecting the temperature sensing device to the cannula.

3. The combined cannula and temperature sensing device according to claim 2, wherein the retaining mechanism further comprises a retaining feature which engages with a portion of the sensor body and assists with retaining the pressure sensing device within the retaining mechanism.

4. The combined cannula and temperature sensing device according to claim 1, wherein the retaining mechanism comprises a pair of spaced apart holsters each having with a passage extending therethrough, and the sensor body has an interference fit with the passage of each of the holsters for releasably connecting the temperature sensing device to the cannula.

5. The combined cannula and temperature sensing device according to claim 1, wherein the at least one temperature sensor comprises a first temperature sensor, a second temperature sensor, and a third temperature sensor, the first temperature sensor is positionable adjacent to a first nasal cavity of the patient, the second temperature sensor is positionable adjacent a second nasal cavity of the patient, and the third temperature sensor is positionable adjacent a mouth of a patient for detecting breathing of the patient.

6. The combined cannula and temperature sensing device according to claim 1, wherein an oral prong communicates with the internal flow chamber and with the pair of nasal prongs.

7. The combined cannula and temperature sensing device according to claim 1, wherein a septum divides the internal flow chamber into a first section and a second section of the internal flow channel and a first one of the pair of nasal prongs communicates with the first section of the internal flow chamber and a second one of the pair of nasal prongs communicates with the second section of the internal flow chamber.

8. The combined cannula and temperature sensing device according to claim 1, wherein a septum divides the internal flow chamber into a first section and a second section of the internal flow channel and a first one of the pair of nasal prongs communicates with the first section of the internal flow chamber and a second one of the pair of nasal prongs communicates with the second section of the internal flow chamber; and the cannula includes at least one oral prong and the at least one oral prong communicates with the second section of the internal flow chamber.

9. The combined cannula and temperature sensing device according to claim 1, wherein an oral prong communicates with the internal flow chamber and with the pair of nasal prongs, and the at least one oral prong has a bow shape which generally extends substantially normal to a centerline of the cannula and then a remote end of the oral prong bends back toward the centerline of the cannula.

10. The combined cannula and temperature sensing device according to claim 1, wherein the cannula body includes first and second oral prongs, the first oral prong communicates with the internal flow chamber while the second oral is sized for receiving and accommodating a malleable member which enables configuring the second oral prong into a desired orientation, relative to the mouth of a patient, for locating a detection opening of the first oral prong to detect breathing of the patient.

11. The combined cannula and temperature sensing device according to claim 1, wherein the cannula body includes first and second oral prongs, a septum divides the internal flow chamber into a first section and a second section of the internal flow channel and a first one of the pair of nasal prongs communicates with the first section of the internal flow chamber and a second one of the pair of nasal prongs communicates with the second section of the internal flow chamber, and a first one of the oral prongs communicates with the first section of the internal flow chamber and a second one of the oral prongs communicates with the second section of the internal flow chamber.

12. The combined cannula and temperature sensing device according to claim 1, wherein the cannula body includes first and second oral prongs, the cannula body defines first and second internal flow chambers, a septum divides the first internal flow chamber into first and second sections of the first flow channel, and a first one of the pair of nasal prongs communicates with the first section of the first internal flow chamber and a second one of the pair of nasal prongs communicates with the second section of the first internal flow chamber, and a first one of the oral prongs communicates with the second internal flow chamber and a second one of the oral prongs receives and accommodates a malleable member which enables the second oral prong to be configured into a desired orientation, relative to the mouth of a patient, for locating a detection opening of the first oral prong to detect breathing of the patient.

13. The combined cannula and temperature sensing device according to claim 1, wherein the cannula body includes first and second oral prongs, the cannula body defines first and second internal flow chambers, a first septum divides the first internal flow chamber into first and second sections of the first flow channel, and a first one of the pair of nasal prongs communicates with the first section of the first internal flow chamber and a second one of the pair of nasal prongs communicates with the second section of the first internal flow chamber, and
a second septum divides the second internal flow chamber into first and second sections of the second flow channel, a first one of the oral prongs communicates with the first section of the second internal flow chamber and a second one of the oral prongs communicates with the second section of the second internal flow chamber.

14. The combined cannula and temperature sensing device according to claim 1, wherein the at least one temperature sensor comprises at least a first temperature sensor and a second temperature sensor supported by the sensor body, and the first temperature sensor is positionable adjacent to a first nasal cavity of the patient while the second temperature sensor is positionable adjacent a second nasal cavity for detecting breathing of the patient.

15. The combined cannula and temperature sensing device according to claim 1, wherein the at least one temperature sensor comprises an oral temperature sensor supported by the sensor body, and the oral temperature sensor extends from the sensor body and is positionable adjacent a mouth of a patient for detecting breathing of the patient.

16. The combined cannula and temperature sensing device according to claim 1, wherein the at least one temperature sensor comprises a first temperature sensor, a second temperature sensor, and a third temperature sensor, and each one of the first temperature sensor, the second temperature sensor, and the third temperature sensor is one of a thermocouple and thermistor.

17. The combined cannula and temperature sensing device according to claim 1, wherein the sensor body comprises a first branch and a second branch, and the first branch and the second branch each form an upward acute angle (B), with respect to a horizontal plane extending through the sensor body, of between 4° and 30°.

18. The combined cannula and temperature sensing device according to claim 1, wherein the sensor body comprises a first branch and a second branch, the first branch and the second branch each form a rearward acute angle (C), with respect to a vertical plane extending through the sensor body, of between about 5° and 40°.

19. The combined cannula and temperature sensing device according to claim 1, wherein the sensor body comprises a first branch and a second branch, and the first branch and the second branch each form an upward acute angle (B), with respect to a horizontal plane extending through the sensor body, of between 4° and 30°; and
the first branch and the second branch each form a rearward acute angle (C), with respect to a vertical plane extending through the sensor body, of between about 5° and 40.

20. The combined cannula and temperature sensing device according to claim 4, wherein the first temperature sensor, the second temperature sensor, and the third temperature sensor are interconnected one of in series and in parallel with one another for detecting breathing of the patient.

21. The combined cannula and temperature sensing device according to claim 1, wherein the retaining mechanism is a holster and the sensor body has an interference fit so as to facilitate secure releasable retention of the temperature sensing device by the cannula, and sensor body has a stop which prevents further insertion of the temperature sensing device into the holster of the cannula; and
the sensor body has a substantially cylindrical section which facilitates supporting the temperature sensing device by the retaining mechanism.

22. The combined cannula and temperature sensing device according to claim 1, wherein the temperature sensing device is one of:
disposed of following use thereof, and
at least one of sterilized and disinfected to permit subsequent reuse of the temperature sensing device.

23. The combined cannula and temperature sensing device according to claim 1, wherein the temperature sensing device generally has a T-shaped configuration and comprises a first temperature sensor positionable adjacent to a first nasal cavity of the patient, a second temperature sensor positionable adjacent a second nasal cavity of the patient, and a third temperature sensor positionable adjacent a mouth of a patient for detecting breathing of the patient.

24. A cannula for detecting breathing of a patient, the cannula comprising:
a cannula body defining at least one internal flow chamber therein;
at least one tube being affixed to the cannula body and communicating with the internal flow chamber;
a pair of nasal prongs communicating with the internal flow chamber, and
the cannula body having retaining mechanism for releasably attaching the temperature sensing device to the cannula;
an oral prong communicates with the internal flow chamber and with the pair of nasal prongs, and the at least one oral prong has a bow shape which generally extends substantially normal to a centerline of the cannula and then bends back toward the centerline of the cannula, and
a remote end of the oral prong has a detection opening formed therein for detecting breathing of the patient.

25. The cannula according to claim 24, in combination with a temperature sensing device comprising a sensor body having first, second and third temperature sensors for detecting breathing of the patient, wherein the cannula body includes first and second oral prongs, the first oral prong communicates with the internal flow chamber while the second oral is sized for receiving and accommodating the third temperature sensor which facilitates configuring the second oral prong into a desired orientation for detecting breathing of the patient.
